(19) United States
(12) Patent Application Publication
(10) Pub. No.: US 2013/0024008 A1
(43) Pub. Date: Jan. 24, 2013

(54) SHOE FOR PROSTHETIC FEET

(71) Applicants: Elisabeth A. Treger, Chicago, IL (US); Bruce L. Miller, JR., Mill Valley, CA (US)

(72) Inventors: Elisabeth A. Treger, Chicago, IL (US); Bruce L. Miller, JR., Mill Valley, CA (US)

(21) Appl. No.: 13/493,886

(22) Filed: Jun. 11, 2012

Related U.S. Application Data
(60) Provisional application No. 61/511,022, filed on Jul. 22, 2011.

(51) Int. Cl. A61F 2/66

(52) U.S. Cl. 623/55

(57) ABSTRACT

A customized shoe for a prosthetic foot includes a polymeric body that is formed to the bottom of the prosthetic foot. The polymeric body may be formed to the bottom of the foot using a molding process or heat and pressure. The polymeric body may provide the function of a sole of the shoe or it may be mediator that is placed between the foot and the shoe sole.

### RELATED APPLICATIONS

This non-provisional patent application claims priority to U.S. Provisional Application Ser. No. 61/511,022, Entitled “SHOE FOR PROSTHETIC FEET”, by Elisabeth A. Treger, filed on 22-JUL.-2011, incorporated herein by references under the benefit of U.S.C. 119(e).

### FIELD OF THE INVENTION

The present subject matter relates generally to footwear for prosthetic feet. More specifically, the present invention relates to a customized shoe and a process for making the customized shoe to provide the most effective vibrotactile association between a prosthetic user and the ground.

### BACKGROUND OF THE INVENTION

Prior lower extremity prosthetics have often included a prosthetic foot shell that can have a similar shape and cosmetic appearance of human feet and serves as an interface between the user and the ground. These foot shells dampen the vibrotactile association of contact between the prosthetic foot, the ground, and the residual limb. Vibrotactile association in this context concerns the ability of a prosthetic foot user to most effectively sense contact of the foot with the ground. The foot shell acts as a deadening zone through which the vibrotactile feedback is lost. This in turn reduces proprioception which concerns a user's sense of body portion orientation and placement. In addition to the adverse effects on user feedback sensations, the foot shells can be bulky, heavy, and require frequent replacement.

The present invention may apply to various types of prosthetic feet including SACH (solid ankle, cushioned heel) prosthetic feet, single axis prosthetic feet, multiple axis prosthetic feet, and ESDR (energy storing dynamic response) prosthetic feet. The ESDR (energy storing dynamic response) category of prosthetic feet is intended for the most active recipients and actually can resiliently store energy. Examples of such feet 2 are illustrated in FIGS. 1-3. The present invention will be described for one of these ESDR feet but it is to be understood that it may apply to various other prosthetic feet that are used for more sedentary lifestyles.

Each of the feet 2 in FIGS. 1-3 are at least partially formed of a strong, resilient material such as a carbon fiber composite. Each foot includes a forward extending toe portion 4 and a rearward extending heel portion 6 that are blade-like in construction. Blade-like in construction refers to a somewhat thin and somewhat sheet-like in construction. Referring to FIG. 1 the toe portion 4 extends downwardly and forwardly and tapers in sheet thickness from an upper portion toward the portion that contacts a walking surface. The heel portion 6 defines a curve shape and also tapers in thickness as it extends in a rearward direction. Referring now to FIG. 2 the toe portion and heel portion 6 are defined by a single sheet of material having a substantially or nearly constant thickness. Finally FIG. 3 depicts a design formed of two sheets of material coupled together including toe portion 4 and heel portion 6.

Because blade-like portion 4 and 6 are generally constructed of carbon fiber composite materials (or the rough equivalent in terms of material properties), they are typically quite expensive. The users of these feet tend to be active users that have a harder heel strike and more active swing phase as they walk relative to users of other designs. As a result the blade-like portions tend to wear out and require replacement. The wear out mode of the feet typically results in discomfort or even injury to a user. Thus there is a need for a foot covering or footwear to protect the feet from excessive wear.

Foot wear issues notwithstanding there is also a need to optimize the interface between the feet and the ground during walking The footwear must help to dissipate the energy from the heel strike and provide stability to prevent repetitive injury to the user. This need is important for ESDR feet because of the active use of the users and the fact that ESDR feet have a minimal amount of material for absorbing shock and dissipating energy. This is also important for less active users.

Prior approaches to this problem have involved enabling the blade-like feet to accommodate standard shoes, e.g., retail shoes for natural feet. This has been accomplished by fitting each ESDR foot prosthesis into a “foot shell” that approximates the shape of a natural foot. The retail shoe is then placed over the foot shell.

The use of a retail shoe over a foot shell has various problematic issues: (1) the foot shell adds more material for sensory input to travel from the earth's surface to the user's body thereby reducing the vibrotactile feedback to the user, (2) the foot shell adds weight and bulk to the prosthetic foot for which there is no benefit, (3) the retail shoe further isolates the user from the ground, and (4) the retail shoe is optimized in design for natural feet, not prosthetics. Thus the end result is far from optimal for the user.

Previously the options for fitting shoes to prosthetic feet have been limited. A need exists for an inexpensive shoe that can more optimally fit onto a prosthetic foot without a foot shell, optimize proprioception, enable more fluid movement for the wearer, provide suitable absorption and energy dissipation and additional spring function at the heel strike that fit snugly around the foot and allow for optimal contact between a walking surface and foot as described and claimed herein.

### BRIEF SUMMARY OF THE INVENTION

The present invention provides a shoe for use with a prosthetic foot that does not require the use of a foot shell. The shoe provided herein stabilizes and supports the heel of the prosthetic foot to accommodate the impact at heel strike, pad and protect the sole of the prosthetic foot and keeps the prosthetic foot in place in the shoe from toe-off to dorsiflexion and throughout entire gait cycles.

In one embodiment the shoe includes a mediator formed to the prosthetic foot and an outer sole between the mediator and a walking surface. In some embodiments the shoe may include a heel pad, a heel stabilizer, and a fastener. The sole protects the bottom of the foot from wear and pads the impact of the wearer's steps. The heel stabilizer secures the foot within the shoe and accommodates the rearward flex of the heel from the impact forces caused by the heel strike. The fastener assists in securing the shoe to the foot and enabling an easy attachment and release of the shoe from the foot. The mediator is provided to occupy any voids between foot and sole and to improve proprioception.

Other embodiments are possible that have fewer than the above elements. For example, in one embodiment the sole and the mediator are combined into one portion. This combined sole/mediator would be formed to the bottom of the foot in order to optimize the interface between the foot and the walking surface. The shoe of the present invention provides benefits to the user that may include any or all of the following.

a. An advantage of the present invention is that a prosthetic foot is provided with an effective interface to the earth's surface that maximizes vibrotactile feedback to the user.

b. The present invention may provide substantial benefits to the users of ESDR (energy storing dynamic response) prosthetic feet.

c. The present invention may benefit various types of prosthetic feet including SACH (solid-ankle, cushioned-heel) prosthetic feet, single-axis prosthetic feet, and multiple-axis prosthetic feet. Other types of prosthetic feet not mentioned may also benefit from the present invention.

d. One advantage of the present shoe is that certain embodiments may be provided at a relatively low cost.

e. A further advantage of the present shoe is that it does not require the use of a foot shell.

f. Yet another advantage of the present shoe is that it may include a heel pad to absorb shock at impact.

g. A further advantage of the present shoe is that it is provided to optimize proprioception. This has the effect of reducing the cognitive burden on the user.

h. Another advantage of the present shoe is that it includes an internal mediator that molds to a prosthetic foot creating a locking effect to keep the shoe on the foot as well as increased sensation to the surface under foot.

i. Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a perspective view of a first embodiment of an ESDR (energy storing dynamic response) foot.

FIG. 2 is a perspective view of a second embodiment of an ESDR (energy storing dynamic response) foot.

FIG. 3 is a perspective view of a first embodiment of an ESDR (energy storing dynamic response) foot.

FIG. 4 is a side perspective view of an exemplary shoe in accordance with the present invention.

FIG. 5 is a cross-sectional view taken from section AA′ of FIG. 4.

FIG. 6 is a cross-sectional view taken from section BB′ of FIG. 4.

FIG. 7 is a flow chart representation of a method for fabricating the shoe of the present invention.

FIG. 8 depicts an embodiment of a second embodiment of a shoe according to the present invention.

FIG. 9A depicts cross section CC′ taken from FIG. 8 in an un-cinched state.

FIG. 9B depicts cross section CC′ taken from FIG. 8 in a cinched state.

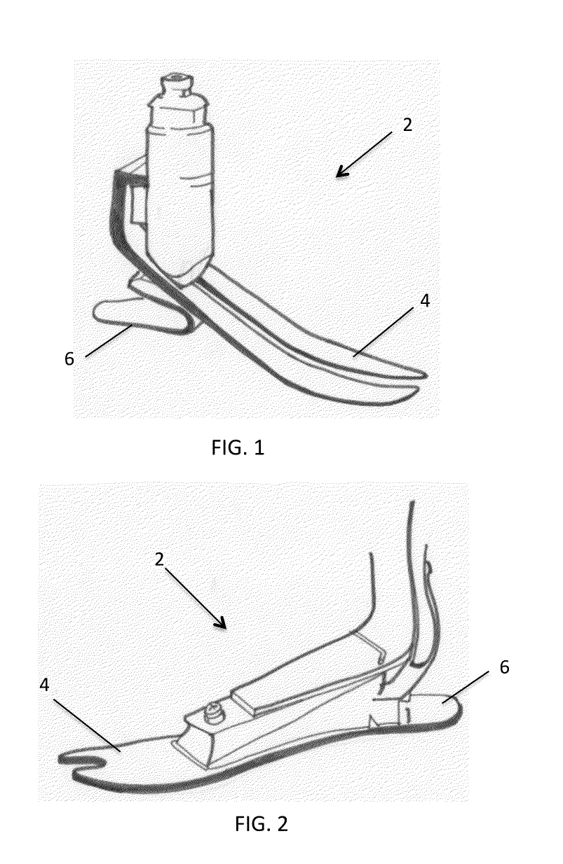

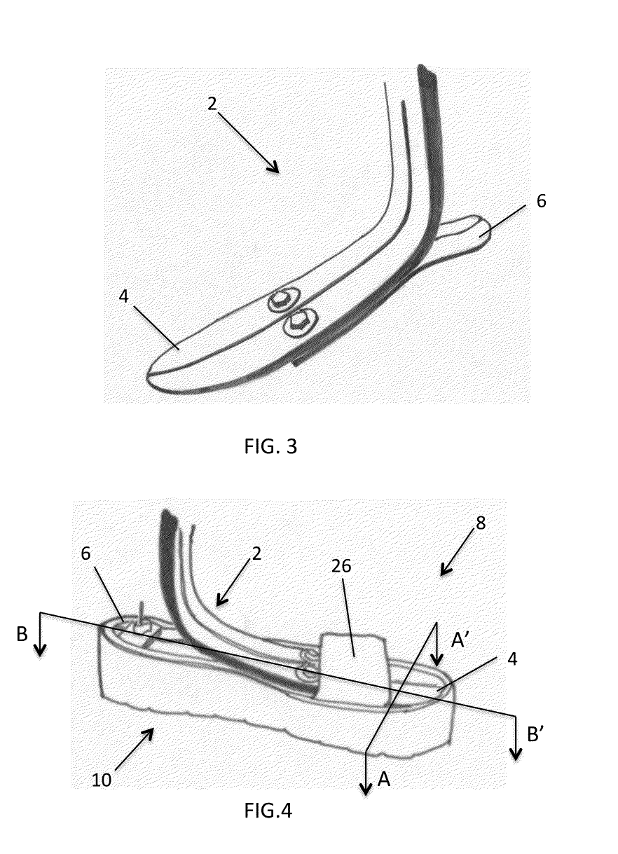

### DETAILED DESCRIPTION OF THE INVENTION

While the present invention is described in use with ESDR (Energy Storing Dynamic Response) prosthetic feet, it is to be understood that this may apply to any number of prosthetic foot designs. These may include SACH (solid ankle cushioned heel) prosthetic feet, single-axis prosthetic feet, and multi-axis prosthetic feet. Also other designs are envisioned. The designs discussed include a polymeric layer that is formed to the bottom of a prosthetic foot. In one embodiment the polymeric layer includes the sole of the footwear. In a second embodiment the polymeric layer is a mediator that is to be fitted with a sole. In yet a third embodiment the polymeric layer is a mediator designed to be fit inside of a standard shoe.

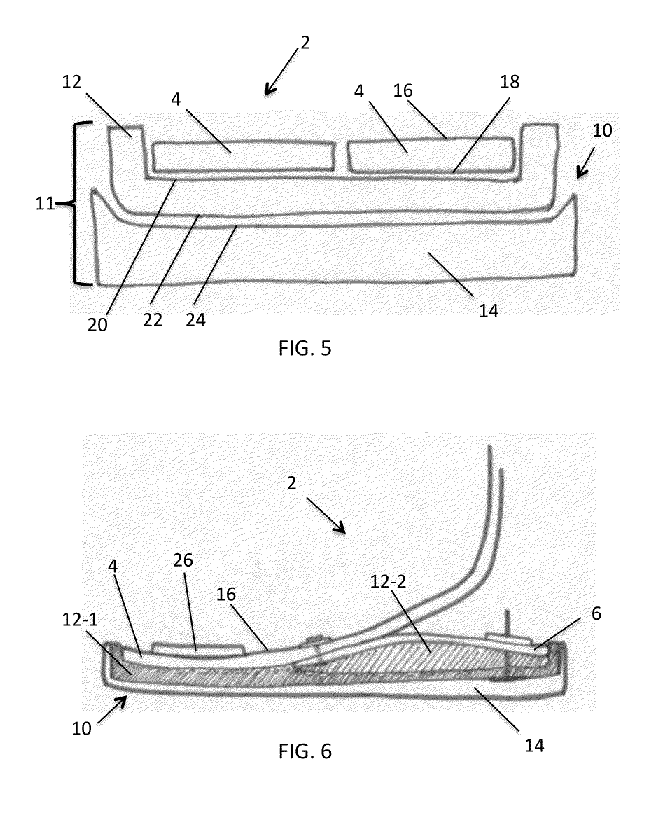

FIG. 4 is a perspective side view depicting a prosthetic support system 8 of the present invention including an ESDR foot 2 and shoe 10. FIGS. 5 is a cross sectional view taken from AA′ of FIG. 4. FIG. 5 depicts a cross section of toe portion 4 of ESDR shoe 2 with portions of shoe 10. Shoe 10 includes a polymer body 11 formed at least partially from a resilient polymeric material. In the illustrated example, polymer body 11 includes mediator 12 and sole 14. Mediator 12 is preferably formed from an elastomeric polymer such as a polyurethane. In some embodiments there may be other material layers not shown that are between mediator 12 and sole 14 such as a cloth layer.

ESDR foot 2 includes an upper surface 16 and a lower surface 18. Mediator 12 is formed onto the lower surface 18 of ESDR foot 2 such that an upper surface 20 of mediator 12 is in intimate contact with the lower surface 18 of ESDR foot. Preferably there is no gap between surfaces 18 and 20. One way to form mediator 12 to lower surface 18 is by using a molding process in which the lower surface 18 of foot 2 defines a molding surface. In one embodiment mediator 12 is formed to lower surface 18 using a vacuum molding process. In another embodiment the mediator 12 is molded separately from ESDR foot 2 and then formed to surface 18 using heat and pressure. The heat and pressure then melts and conforms mediator 12 to lower surface 18. Other processes are possible but preferably the surface 20 of mediator closely matches lower surface 18 of foot 2.

The result is an interface between mediator 12 and surface 18 with essentially no gaps. A lower surface 22 of mediator 12 is attached to an upper surface 24 of sole 14. It is preferable that there is intimate contact between surfaces 22 and 24 such that no gaps exist. One way to accomplish this is to use processes such as welding or gluing to attach surfaces 22 and 24. Without any gaps in the interfaces between ESDR foot 2 and mediator 12 or between mediator 12 and sole 14, stability of shoe system 8 is maximized and the user obtains an effective vibrotactile feedback with a walking or running surface during use of shoe system 8.

FIG. 6 depicts cross section BB' taken along the long axis of the prosthetic support system 8. In this figure mediator 12 includes two portions including first portion 12-1 that extends under the entire ESDR foot 2 and second portion 12-2 that is located under the heel portion 6 of ESDR foot 2. The first portion 12-1 is formed from a first polymer and the second portion 12-2 is formed from a second polymer. In an exemplary embodiment the first polymer (12-1) has a lower elastic modulus than the second polymer (12-2).

Referring to FIGS. 4 and 6, a fastener 26 couples foot 2 to shoe 10. FIGS. 4 and 6 depict fastener 26 as extending over only a portion of upper surface 16 of foot 2 but it is to be understood that support may extend over other portions or the entirety of the upper surface 16 of foot 2 in various embodiments depending on support requirements and preferences for shoe 10. Fastener 26 may come in varying forms such as belt buckles, drawstrings, laces, Velcro trips, and other means for coupling a lower portion of shoe 10 to foot 2.

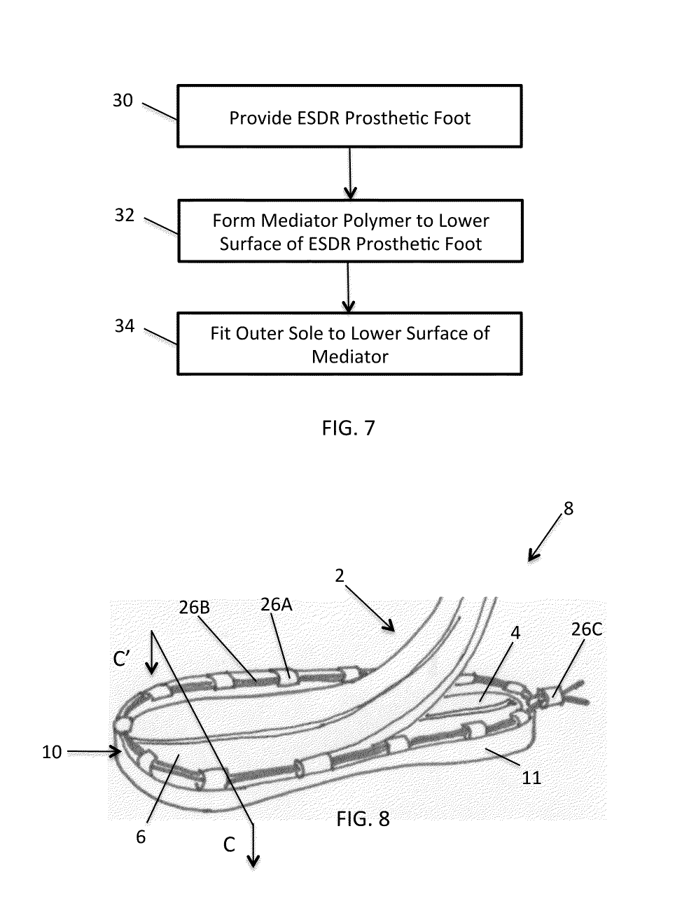

FIG. 7 is a flow chart representation of a process for fabricating the shoe of the present invention. According to step 30, an ESDR foot 2 is provided for the purpose of fitting a shoe according to the present invention. The ESDR foot 2 includes a toe portion 4 and a heel portion 6 which are blade-like portions. The foot 2 has an upper surface 16 lower surface 18.

According to step 32 a body of resilient polymer is formed to the lower surface 18 of the foot 2 to form mediator 12. There are various methods of forming the polymer to lower surface 18. In a first embodiment of step 32 the polymer is molded to surface 18. In this first embodiment, the surface 18 forms a portion of a mold surface. The surface 18 thereby defines an upper surface 20 of mediator 12. Other portions of a mold define a lower surface 22 of mediator 12. In some embodiments a “vacuum molding” process may be used to improve conformance of surfaces 18 and 20 and to eliminate gas or air pockets that may reduce the quality of the contact between surfaces 18 and 20.

In a second embodiment of step 32 a polymer body is placed into contact with surface 18. Heat and pressure is applied to the polymer body to conform the polymer body to the surface 18. This heat and pressure may be applied in conjunction with the application of a vacuum to eliminate air pockets and improve conformance of surfaces 18 and 20.

The resultant mediator 12 formed according to step 32 has an upper surface 20 and a lower surface 22. According to step 34 an outer sole 14 is fitted to the mediator 12. An upper and/or inside surface 24 of the sole fits to lower and/or outer surface 22 of the mediator. In one embodiment surfaces 24 and 22 may be welded together. In a second embodiment surfaces 24 and 22 may be glued together.

FIG. 8 is a perspective view of a simplified embodiment of foot support system 8 that eliminates having a separate mediator 12 and sole 14. In this alternative embodiment the polymer body 11 may be a single resilient polymer material. Shoe 10 also includes a fastener 26 that includes loops 26A, drawstring 26B, and cinch 26C. Drawstring 26B passes through loops 26A and drawstring 26C. Shoe 10 is secured or cinched to foot 2 by tightening drawstring 26B which is secured by cinch 26C.

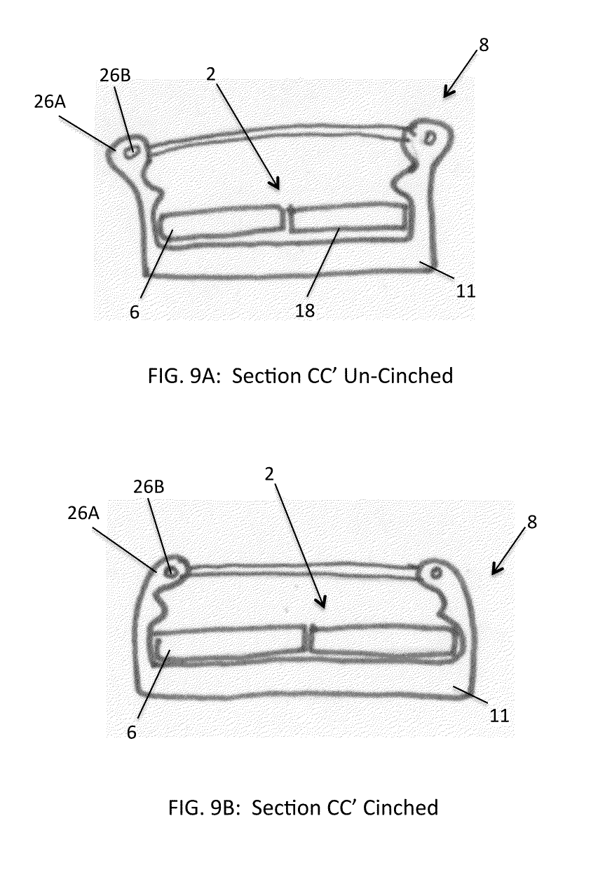

FIG. 9A and 9B are cross sectional views taken through CC′ of FIG. 8 depicting system 8 in an un-cinched (9A) and cinched (9B) state. An advantage of this embodiment of shoe 10 is its extreme simplicity while providing a minimal interface between foot 2 and a walking surface. Like mediator 12 in an earlier embodiment, the polymer body 11 is formed directly to a bottom surface 18 of foot 2.

As with mediator 12, polymer body 11 may be formed to surface 18 via a molding process or using heat and pressure. In this way, polymer body 11 conforms to surface 18. The molding process used may be a vacuum molding process.

Polymer body 11 may be removed from foot 2 as needed. When it is attached to foot surface 18, draw string 26B may be used to secure the polymer body 11 to the foot 2. The action of tightening drawstring 26B through cinch 26C has the effect of fastening polymer body 11 to foot 2.

It should be noted that various changes and modifications to the presently preferred embodiments described herein will be apparent to those skilled in the art. Such changes and modifications may be made without departing from the spirit and scope of the present invention and without diminishing its attendant advantages.

What we claim is:

1. A prosthetic support system comprising:
a prosthetic foot having a lower surface and an upper surface;
a polymer body having an upper surface and a lower surface, the upper surface is formed onto the lower surface of the prosthetic foot, the lower surface for contacting a walking surface.

2. The prosthetic support system of claim 1 wherein the prosthetic foot is an ESDR (energy storing dynamic response) foot having a lower blade-like portion that is composed of a resilient strong material.

3. The prosthetic support system of claim 2 wherein the resilient strong material is a carbon fiber composite.

4. The prosthetic support system of claim 1 wherein the prosthetic foot is one of a SACH (solid-ankle, cushioned-heel) prosthetic foot, a single axis prosthetic foot, and a multi-axis prosthetic foot.

5. The prosthetic support system of claim 1 wherein the upper surface is formed onto the lower surface of the prosthetic foot using a molding process whereby the lower surface of the prosthetic foot forms a portion of a molding surface.

6. The prosthetic support system of claim 1 wherein the upper surface of the polymer body is formed onto the lower surface of the prosthetic foot by applying heat and pressure to at least a portion of the polymer body.

7. The prosthetic support system of claim 1 wherein the polymer body includes a mediator and a sole, the mediator having an upper surface that defines the upper surface of the polymer body and a lower surface, the sole having an upper surface that receives the lower surface of the mediator and a lower surface for contacting a walking surface.

8. The prosthetic support system of claim 1 wherein the mediator is formed from two different polymers including a first polymer and a second polymer having a higher elastic modulus than the first polymer.

9. The prosthetic support system of claim 1 further comprising a fastener that couples the prosthetic foot to the sole.

10. A shoe for a prosthetic foot having an upper and lower surface formed by the following steps:
forming an upper surface of a polymer body to the lower surface of the prosthetic foot such that the upper surface of the polymer body replicates the shape of the lower surface of the prosthetic foot; and
attaching a fastening portion to the upper surface of the prosthetic foot such that the fastening portion secures the polymer body to the prosthetic foot.

11. The shoe of claim 10 wherein forming the upper surface of the polymer body to the lower surface of the prosthetic foot includes a molding process whereby the lower surface of the prosthetic foot forms part of a molding surface.

12. The shoe of claim 10 wherein forming the upper surface of the polymer body to the lower surface of the prosthetic foot includes applying heat and pressure to the polymer body to conform the polymer body to the lower surface of the prosthetic foot.

13. The shoe of claim 10 wherein the fastening portion is integral with the polymer body.

14. The shoe of claim 10 wherein the polymer body includes a lower surface and further including the step of fitting the lower surface into a sole.

15. A shoe for a prosthetic foot blade, the foot blade having a lower surface and an upper surface, the shoe comprising:
a polymer body having an upper surface and a lower surface, the upper surface of the polymer body having been formed to the lower surface of the foot blade, the lower surface of the polymer body for contacting a walking surface; and
a fastening portion coupling the polymer body to the foot blade.

16. The shoe of claim 15 wherein the polymer body is formed from a single resilient polymer.

17. The shoe of claim 15 wherein the polymer body is formed to the lower surface of the foot blade using heat and pressure.

18. The shoe of claim 15 wherein the polymer body is formed to the lower surface of the foot blade using a molding process.

19. The shoe of claim 15 wherein polymer body includes a mediator and a sole each having upper and lower surfaces, the upper surface of the mediator defining the upper surface of the polymer body, the lower surface of the sole for contacting the walking surface, the lower surface of the mediator fitting into the upper surface of the sole.
